(19) United States
(12) Patent Application Publication
(10) Pub. No.: US 2021/0186805 A1
(43) Pub. Date: Jun. 24, 2021

(54) CARDIOPULMONARY RESUSCITATION SYSTEM

(71) Applicant: KOHKEN MEDICAL CO., LTD., Bunkyo-ku, Tokyo (JP)

(72) Inventors: Eiichi MATSUI, Bunkyo-ku, Tokyo (JP); Mitsuo MATSUI, Bunkyo-ku, Tokyo (JP); Yukio SAITO, Bunkyo-ku, Tokyo (JP); Hitoshi TAZAKI, Bunkyo-ku, Tokyo (JP)

(21) Appl. No.: 16/078,115

(22) PCT Filed: Mar. 3, 2017
(86) PCT No.: PCT/JP2017/008420

§ 371 Date: Aug. 21, 2018

(30) Foreign Application Priority Data
Mar. 8, 2016 (JP) 2016-044363

(51) Int. Cl. A61H 31/00; A61M 16/00

(52) U.S. Cl. CPC A61H 31/006; A61M 16/024; A61H 2201/107; A61H 2230/425; A61M 2230/42; A61H 2201/1246; A61H 2201/1207; A61M 2205/05; A61M 2205/3334; A61H 2201/5007

(57) ABSTRACT

A cardiopulmonary resuscitation system capable of avoiding fighting in an asynchronous mode in which sternum compression and artificial respiration are performed independently and continuously. The cardiopulmonary resuscitation system includes: a sternum compressor that includes an impact hammer for compressing the chest of a patient and repeats a sternum compression cycle having, as one cycle, a compression period in which the impact hammer is pressed against the chest and a recoil period in which the impact hammer is separated from the chest; an artificial respirator that repeats an artificial respiration cycle having, as one cycle, an inhalation period in which respiratory gas is supplied to the patient and an exhalation period in which supply of the respiratory gas is stopped; and a controller that controls the artificial respirator and the sternum compressor, the controller executes the artificial respiration cycle a predetermined number of times per unit time while executing the sternum compression cycle a predetermined number of times per unit time, and stops pressing the impact hammer against the chest during the compression period overlapping with the inhalation period.

### BACKGROUND

### 1. Field of the Disclosure

The present disclosure relates to a cardiopulmonary resuscitation system.

### 2. Discussion of the Background Art

As a cardiopulmonary resuscitation (also referred to as CPR) method, a method for combining sternum compression with hands and mouth-to-mouth artificial respiration is known. However, it is difficult to manually perform stable and high-quality cardiopulmonary resuscitation. Therefore, a cardiopulmonary resuscitator for automatically performing sternum compression and artificial respiration has been proposed. For example, the present applicant has proposed an automatic cardiopulmonary resuscitator for performing cardiac massage by repeatedly applying an impact at adjusted regular intervals and supplying respiratory gas for ventilation at adjusted timing and duration (see, for example, Patent Literature 1).

### CITATION LIST

### Patent Literature

Patent Literature 1: JP 2000-84028 A

### SUMMARY

### Technical Problem

Guidelines 2015 (2015 AHA Guidelines for CPR and ECC) for AHA cardiopulmonary resuscitation and emergency cardiovascular treatment (hereinafter also referred to as Guidelines) specify that “After tracheal intubation, sternum compression and artificial respiration are asynchronous, and continuous sternum compression is performed. Sternum compression is performed at least 100 times per minute, and artificial respiration is performed about 10 times per minute to avoid hyperventilation. In a case where a supraglottic respiratory tract device (such as a laryngeal mask) is used, continuous sternum compression may be performed only in a case where adequate ventilation is possible.”, “It is a condition for success not only in basic life support (BLS) but also in advanced life support (ALS) that continuous and efficient sternum compression is performed. Be careful with a procedure of ALS and judgment to avoid degradation of CPR quality and interruption.”, and “Interruption of sternum compression should be avoided as much as possible also in ALS, and it is inevitable to interrupt sternum compression only in a case where artificial respiration is performed, in a case where electrocardiogram (ECG) or return of spontaneous circulation (ROSC) is evaluated, and in a case where an electric shock is applied”. In addition, Guidelines recommend that compression is performed at 100 times/minute or more and the compression depth for an adult is set to 5 cm or more for high-quality sternum compression.

CPR includes a synchronous mode in which sternum compression and artificial respiration are alternately performed at a predetermined ratio and an asynchronous mode in which sternum compression and artificial respiration are performed independently and continuously. In the asynchronous mode, sternum compression is continuously performed, and a phenomenon (also referred to as fighting) in which the timing of sternum compression and the timing of artificial respiration are coincident and exhalation of a patient and inhalation by an artificial respirator occur simultaneously occurs. When fighting occurs, a risk such as reduction in cardiac output or coronary perfusion pressure, damage to an alveolus due to a high respiratory tract internal pressure, occurrence of traumatic pneumothorax, or reduction in effective alveolar ventilation amount is expected. Guidelines assume CPR based on a method with hands, and even artificial respiration is performed with hands during sternum compression, the ventilation amount is not increased due to resistance of compression, and therefore a big problem hardly occurs. Meanwhile, in CPR with an apparatus, sternum compression and artificial respiration are surely performed, and therefore a risk due to fighting is increased.

Therefore, an object of the present disclosure is to provide a cardiopulmonary resuscitation system capable of avoiding fighting in an asynchronous mode in which sternum compression and artificial respiration are performed independently and continuously.

### Solution to Problem

A cardiopulmonary resuscitation system according to the present disclosure includes: a sternum compression means that includes an impact hammer for compressing the chest of a patient and repeats a sternum compression cycle having, as one cycle, a compression period in which the impact hammer is pressed against the chest and a recoil period in which the impact hammer is separated from the chest; an artificial respiration means that repeats an artificial respiration cycle having, as one cycle, an inhalation period in which respiratory gas is supplied to the patient and an exhalation period in which supply of the respiratory gas is stopped; and a control means that controls the artificial respiration means and the sternum compression means, and is characterized in that the control means executes the artificial respiration cycle a predetermined number of times per unit time while executing the sternum compression cycle a predetermined number of times per unit time, and stops pressing the impact hammer against the chest during the compression period overlapping with the inhalation period.

In the cardiopulmonary resuscitation system according to the present disclosure, in a case where the inhalation period is started during the recoil period, the control means preferably extends the recoil period executed at the start time of the inhalation period at least until the inhalation period ends. Fighting can be avoided while the number of times of artificial respiration is secured.

In the cardiopulmonary resuscitation system according to the present disclosure, in a case where the inhalation period is started during the compression period and the start time of the inhalation period is in the first half period obtained by temporally dividing the compression period into two equal parts, the control means preferably hastens start of the inhalation period by the same time as the time from the start time of the compression period overlapping with the inhalation period to the start time of the inhalation period. Fighting can be avoided even in a case where the inhalation period is started during the compression period.

In the cardiopulmonary resuscitation system according to the present disclosure, in a case where the inhalation period is started during the compression period and the start time of the inhalation period is in the second half period obtained by temporally dividing the compression period into two equal parts, the control means preferably delays start of the inhalation period by the same time as the time from the start time of the inhalation period to the end time of the compression period overlapping with the inhalation period. Fighting can be avoided even in a case where the inhalation period is started during the compression period.

In the cardiopulmonary resuscitation system according to the present disclosure, it is preferable that the control means restarts the sternum compression cycle a predetermined time after the end of the inhalation period, and the sternum compression cycle restarted is started from the compression period. By securing the exhalation time after the end of the inhalation period, it is possible to prevent an intrathoracic pressure from becoming too high.

In the cardiopulmonary resuscitation system according to the present disclosure, the artificial respiration means and the sternum compression means are preferably configured as an integral device. Portability is improved, and cardiopulmonary resuscitation can be quickly started in an early stage of critical care.

In the cardiopulmonary resuscitation system according to the present disclosure, the artificial respiration means and the sternum compression means are preferably configured as individual devices. Cardiopulmonary resuscitation can be performed with higher accuracy.

In the cardiopulmonary resuscitation system according to the present disclosure, the control means is preferably mounted on a device constituting the artificial respiration means. The sternum compression means can be small and lightweight, and sternum compression can be quickly started in an early stage of critical care.

In the cardiopulmonary resuscitation system according to the present disclosure, it is preferable that the sternum compression means includes an elevating means for vertically reciprocating the impact hammer, and a driving system of the elevating means is a gas driving system, an electric driving system, or a mixed driving system of the gas driving system and the electric driving system. By adopting the gas driving system, the setting width of the sternum compression depth can be wide, and sternum compression by a method with hands can be reproduced. By adopting the electric driving system, a device can be simpler.

The present disclosure can provide a cardiopulmonary resuscitation system capable of avoiding fighting in an asynchronous mode in which sternum compression and artificial respiration are performed independently and continuously.

### BRIEF DESCRIPTION OF DRAWINGS

FIG. 1 is a perspective view of an example of a cardiopulmonary resuscitation system according to the present embodiment.

FIG. 2 is a timing chart of a sternum compression cycle and an artificial respiration cycle in a synchronous mode, FIG. 2(a) illustrates a compression waveform, and FIG. 2(b) illustrates a ventilation waveform.

FIG. 3 is a timing chart of a sternum compression cycle and an artificial respiration cycle in an asynchronous mode, FIG. 3(a) illustrates a compression waveform, and FIG. 3(b) illustrates a ventilation waveform.

FIG. 4 is a first example of a timing chart of a sternum compression cycle and an artificial respiration cycle, FIG. 4(a) illustrates a compression waveform based on setting, FIG. 4(b) illustrates a ventilation waveform based on setting, FIG. 4(c) illustrates a compression waveform when fighting is avoided, and FIG. 4(d) illustrates a ventilation waveform when fighting is avoided.

FIG. 5 is a second example of the timing chart of a sternum compression cycle and an artificial respiration cycle, FIG. 5(a) illustrates a compression waveform based on setting, FIG. 5(b) illustrates a ventilation waveform based on setting, FIG. 5(c) illustrates a compression waveform when fighting is avoided, and FIG. 5(d) illustrates a ventilation waveform when fighting is avoided.

FIG. 6 is a third example of the timing chart of a sternum compression cycle and an artificial respiration cycle, FIG. 6(a) illustrates a compression waveform based on setting, FIG. 6(b) illustrates a ventilation waveform based on setting, FIG. 6(c) illustrates a compression waveform when fighting is avoided, and FIG. 6(d) illustrates a ventilation waveform when fighting is avoided.

FIG. 7 is a fourth example of the timing chart of a sternum compression cycle and an artificial respiration cycle, FIG. 7(a) illustrates a compression waveform based on setting, and FIG. 7(b) illustrates a ventilation waveform based on setting.

FIG. 8 is a conceptual diagram of another example of the cardiopulmonary resuscitation system according to the present embodiment.

### DETAILED DESCRIPTION OF THE PREFERRED EMBODIMENT

Next, the present disclosure will be described in detail by describing embodiments, but the present disclosure is not construed as being limited to description thereof. As long as an effect of the present disclosure is exhibited, the embodiments may be modified variously.

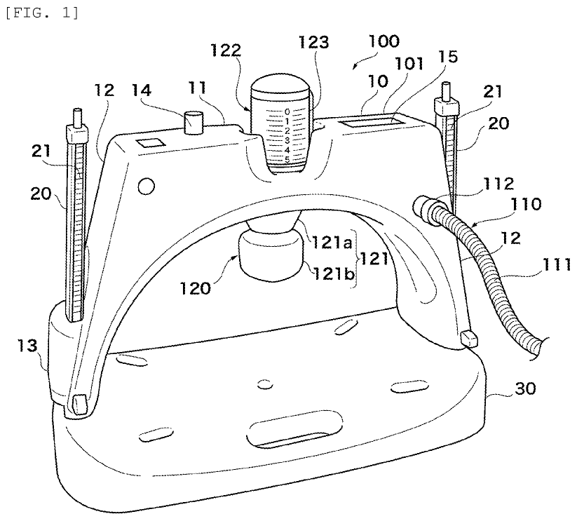

FIG. 1 is a perspective view of an example of a cardiopulmonary resuscitation system according to the present embodiment. As illustrated in FIG. 1, a cardiopulmonary resuscitation system 100 according to the present embodiment includes: a sternum compression means 120 that includes an impact hammer 121 for compressing the chest of a patient and repeats a sternum compression cycle having, as one cycle, a compression period in which the impact hammer 121 is pressed against the chest and a recoil period in which the impact hammer 121 is separated from the chest; an artificial respiration means 110 that repeats an artificial respiration cycle having, as one cycle, an inhalation period in which respiratory gas is supplied to the patient and an exhalation period in which supply of the respiratory gas is stopped; and a control means (not illustrated) that controls the artificial respiration means 110 and the sternum compression means 120, in which the control means executes the artificial respiration cycle a predetermined number of times per unit time while executing the sternum compression cycle a predetermined number of times per unit time, and stops pressing the impact hammer 121 against the chest during the compression period overlapping with the inhalation period.

In the cardiopulmonary resuscitation system 100, as illustrated in FIG. 1, the artificial respiration means 110 and the sternum compression means 120 are preferably configured as an integral device. The cardiopulmonary resuscitation system 100 illustrated in FIG. 1 is an automatic cardiopulmonary resuscitator equipped with an artificial respiration function and a sternum compression function. Portability is favorable, and cardiopulmonary resuscitation can be quickly started in an early stage of critical care.

The artificial respiration means 110 is an artificial respiration unit of the cardiopulmonary resuscitator 100. As illustrated in FIG. 1, the artificial respiration means (hereinafter also referred to as a first artificial respiration unit) 110 includes a hose 111 for injecting respiratory gas into a patient and a gas supply system (not illustrated) for supplying respiratory gas to the hose 111. One end of the hose 111 is connected to a hose insertion port 112 disposed in a housing 101 of the cardiopulmonary resuscitator 100. The other end of the hose 111 is connected to a mask (not illustrated) attached to a patient or a tracheal intubation tube (not illustrated). The gas supply system of the first artificial respiration unit 110 includes a gas supply source such as a gas cylinder or an air tank and piping connecting the gas supply source to the hose 111. In the middle of the piping, for example, a ventilation decompressor for decreasing the pressure of driving gas to a pressure suitable for respiration, a ventilation solenoid valve for supplying respiratory gas to the hose 111 and stopping respiratory gas from the hose 111, and a respiratory tract internal pressure sensor for detecting the pressure in the piping between the ventilation solenoid valve and the hose insertion port 112 are disposed. The respiratory gas is, for example, pure oxygen, oxygen-enriched air, or air.

The sternum compression means 120 is a sternum compression unit of the cardiopulmonary resuscitator 100. As illustrated in FIG. 1, the sternum compression means (hereinafter also referred to as a sternum compression unit) 120 includes an arch portion 10, a vertical rod 20, and a back plate 30.

The arch portion 10 has a top surface portion 11 and left and right side surface portions 12 and is disposed so as to extend over the chest of a patient. The arch portion 10 includes the impact hammer 121 projecting downward from the top surface portion 11 and movably supported in the vertical direction by the top surface portion 11 and an elevating means (elevating mechanism) 122 for vertically reciprocating the impact hammer 121. The impact hammer 121 includes an impact hammer rod 121a connected to the elevating means 122 and an impact head pad 121b attached to a lower end of the impact hammer rod 121a and pressed against the chest of a patient. In a case where the driving system of the elevating means 122 is a gas driving system, the elevating means 122 includes a cylinder 123. The cylinder 123 has a container shape and has a gas supply port (not illustrated) and a gas discharge port (not illustrated). A piston (not illustrated) and a spring (not illustrated) for pushing back the piston at the time of discharge are disposed in an internal space of the cylinder 123.

The driving system (not illustrated) of the elevating means 122 includes a driving gas supply source such as a gas cylinder or an air tank and piping connecting the driving gas supply source to the cylinder 123. In the middle of the piping, for example, a compression depth adjuster for adjusting the stroke width of the vertical reciprocating motion of the elevating means 122 and a compression solenoid valve for supplying driving gas into the cylinder 123 and discharging driving gas from the cylinder 123 are disposed. The driving gas supply source preferably serves also as a gas supply source of the artificial respiration unit 110. The compression solenoid valve is, for example, a three-way solenoid valve.

A part of the arch portion 10 is preferably the housing 101. The housing 101 houses the gas supply system of the first artificial respiration unit 110, the driving system for driving the elevating means 122, the control means of the cardiopulmonary resuscitator 100, and the like.

A pair of vertical rods 20 is disposed on the left and right sides and is fixed to fixing portions 13 disposed at lower ends of the left and right side surface portions 12 of the arch portion, respectively. For example, the vertical rod 20 is engaged with a ratchet of the fixing portion 13 to support the arch portion 10 so as to be movable in the vertical direction. As illustrated in FIG. 1, the vertical rod 20 preferably has a scale 21 displayed. Preferably, the arch portion 10 is pushed down toward the chest of a patient while the arch portion 10 is set on the patient, the scale is read when the impact head pad 121b comes into contact with the chest of the patient, and the read scale is recorded as the chest thickness of the patient. Based on this read sternum thickness, the compression depth of the impact hammer 121 can be set. In this way, finer adjustment of the compression depth suitable for each patient can be performed.

The back plate 30 is a plate for supporting a lower surface of the chest of a patient. For example, by engaging an engagement portion (not illustrated) such as a groove or a hole formed in the back plate 30 with a projection (not illustrated) formed at a lower end of the vertical rod 20, the back plate 30 is fixed to the arch portion 10 detachably.

The control means (hereinafter also referred to as a first control unit) is, for example, a printed circuit board. The first control unit controls the gas supply system of the first artificial respiration unit 110 and the driving system of the elevating means 122.

The first control unit closes the ventilation solenoid valve in a case where a pressure detected by the respiratory tract internal pressure sensor is equal to or higher than a predetermined pressure. It is thereby possible to prevent injection of high pressure gas into a patient.

The first control unit adjusts the frequency (number of times of ventilation) of the artificial respiration cycle by the first artificial respiration unit 110. Here, the number of times of ventilation is the number of times for performing the artificial respiration cycle in one minute, for example, 6 to 20 times/minute. The flow rate of respiratory gas by the first artificial respiration unit 110 is fixed to, for example, 24 liters/minute. The ventilation amount of respiratory gas is, for example, 200 to 600 ml/time. The length of the inhalation period is, for example, 0.5 to 1.5 seconds. The lengths of the inhalation period and the exhalation period vary depending on the flow rate of respiratory gas, the number of times of ventilation, and the ventilation amount. For example, in a case where the flow rate of respiratory gas is 24 liters/minute, the number of times of ventilation is set to 10 times/minute, and the ventilation amount is set to 200 ml/time, the inhalation period is 0.5 seconds, and the exhalation period is 5.5 seconds.

The first control unit controls the compression solenoid valve to supply driving gas into the cylinder 123 or discharge the driving gas from the inside of the cylinder 123. When the driving gas is supplied into the cylinder 123, a piston is pushed down against a repulsive force of a spring, and the impact hammer 121 moves downward. When the driving gas is discharged from the inside of the cylinder 123, the spring expands, the piston is pushed up, and the impact hammer 121 moves upward. By repeating these, the impact hammer 121 reciprocates vertically.

In addition, the first control unit adjusts the frequency of the sternum compression cycle (the number of times of compression) and the stroke width of the vertical reciprocating motion of the elevating means 122. The stroke width is switched, for example, by turning of an adjustment knob 14 by an operator. Here, the number of times of compression is the number of times for performing the sternum compression cycle in one minute, and Guidelines recommend that the number of times of compression is 100 times/minute or more. A compression period and a recoil period preferably have the same length as each other. For example, in a case where the number of times of compression is 100 times/minute, the time per sternum compression cycle is 0.6 seconds, and the compression period and the recoil period are each 0.3 seconds. The stroke width of the vertical reciprocating motion is appropriately adjusted according to a patient, but Guidelines recommend that the stroke width is 5 cm or more in an adult.

The cardiopulmonary resuscitator 100 preferably includes a mode switching unit 15. The mode switching unit 15 is a switch for switching an operation timing mode of each of the first artificial respiration unit 110 and the sternum compression unit 120. The mode switching unit 15 is, for example, a panel as illustrated in FIG. 1 or a knob (not illustrated). Although FIG. 1 illustrates an example in which the mode switching unit 15 is disposed in the arch portion 10, the present disclosure is not limited thereto, and the mode switching unit 15 may be disposed on the back plate 30, for example.

The operation timing mode of each of the first artificial respiration unit 110 and the sternum compression unit 120 is roughly divided into a synchronous mode and an asynchronous mode.

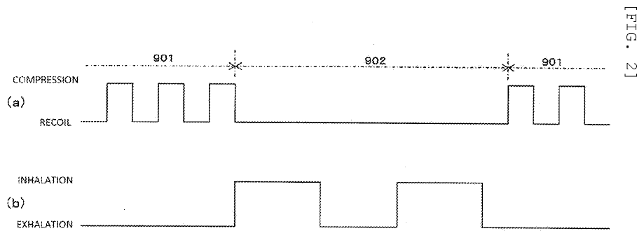

FIG. 2 is a timing chart of a sternum compression cycle and an artificial respiration cycle in a synchronous mode, FIG. 2(a) illustrates a compression waveform, and FIG. 2(b) illustrates a ventilation waveform. Here, in the timing chart, a compression waveform at the height of “compression” indicates that the compression period is being executed, and a compression waveform at the height of “recoil” indicates that the recoil period is being executed. A ventilation waveform at the height of “inhalation” indicates that the inhalation period is being executed, and a ventilation waveform at the height of “exhalation” indicates that the exhalation period is being executed. In the synchronous mode, as illustrated in FIG. 2, the first control unit performs control to alternately execute an execution period 901 in which the sternum compression cycle is executed a predetermined number of times per unit time and a standby period 902 in which execution of the sternum compression cycle is temporarily stopped while the impact hammer 121 is separated from the chest, and executes the artificial respiration cycle a predetermined number of times per unit time during the standby period 902.

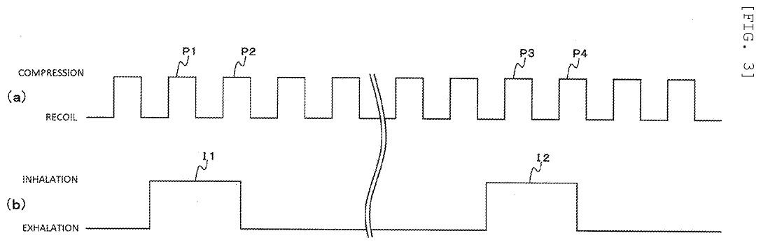

FIG. 3 is a timing chart of a sternum compression cycle and an artificial respiration cycle in an asynchronous mode, FIG. 3(a) illustrates a compression waveform, and FIG. 3(b) illustrates a ventilation waveform. In the asynchronous mode, as illustrated in FIG. 3, the first control unit executes the artificial respiration cycle a predetermined number of times per unit time while executing the sternum compression cycle a predetermined number of times per unit time.

In FIG. 3, the entire compression period P1 and a part of the compression period P2 overlap with the inhalation period I1, and the entire compression period P3 and a part of the compression period P4 overlap with the inhalation period I2. Fighting occurs in these overlapping periods. In the present embodiment, when the number of times of compression, the number of times of ventilation, and the ventilation amount are set, in a case where the sternum compression cycle and the artificial respiration cycle are executed based on the setting, the first control unit calculates a compression period overlapping with an inhalation period in advance and shifts the timing of the compression period or the inhalation period such that the inhalation period and the compression period do not overlap with each other. This avoids fighting. Here, the asynchronous mode in the present disclosure for avoiding fighting may be referred to as an “avoidance type asynchronous mode” in order to distinguish the asynchronous mode in the present disclosure from a conventional asynchronous mode in which fighting occurs.

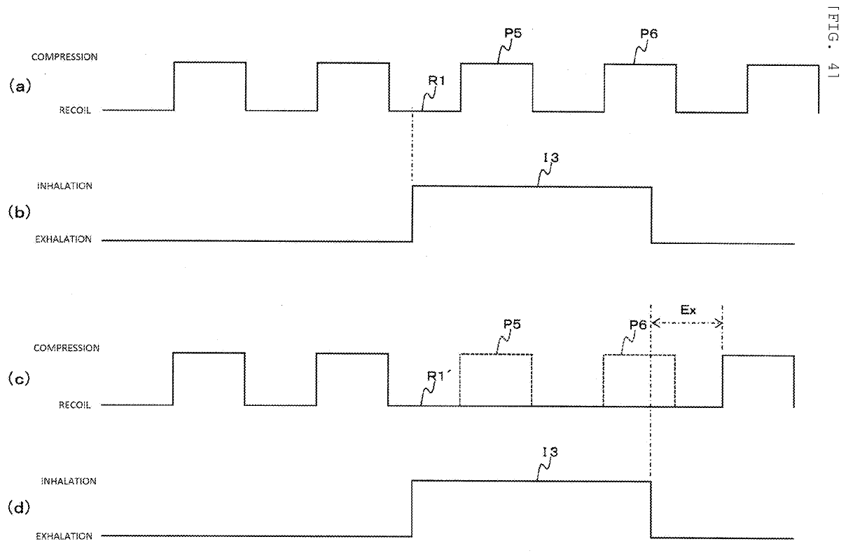

The avoidance type asynchronous mode is roughly divided into three patterns depending on whether the start time of the inhalation period is during the recoil period (first example), during the first half period of the compression period (second example), or during the second half period of the compression period (third example). Next, a specific example of avoiding fighting will be described with reference to FIGS. 4 to 6.

### First Example

FIGS. 4(a) and 4(b) are waveforms in a case where the sternum compression cycle and the artificial respiration cycle are executed based on setting of the number of times of compression, the number of times of ventilation, and the length of the inhalation period. In FIGS. 4(a) and 4(b), the inhalation period I3 is started during the recoil period R1. In this state, the entire compression period P5 and a part of the compression period P6 overlap with the inhalation period I3. In this case, as illustrated in FIGS. 4(c) and 4(d), the first control unit extends the recoil period R1′ executed at the start time of the inhalation period I3 at least until the inhalation period I3 ends. As a result, overlapping between the inhalation period I3 and the compression periods P5 and P6 is eliminated, and fighting can be avoided. In a case where the entire compression period P5 overlaps with the inhalation period I3, the first control unit cancels the entire compression period P5 (indicated by a dotted line in FIG. 4(c)). In a case where a part of the compression period P6 overlaps with the inhalation period I3, the first control unit preferably cancels the entire compression period P6 (indicated by a dotted line in FIG. 4(c)). Alternatively, the first control unit may cancel only a period overlapping with the inhalation period I3 in the compression period P6. In the first example, the number of times of compression is smaller than a set value by the amount overlapping with the inhalation period, and the number of times of ventilation is the same as a set value.

### Second Example

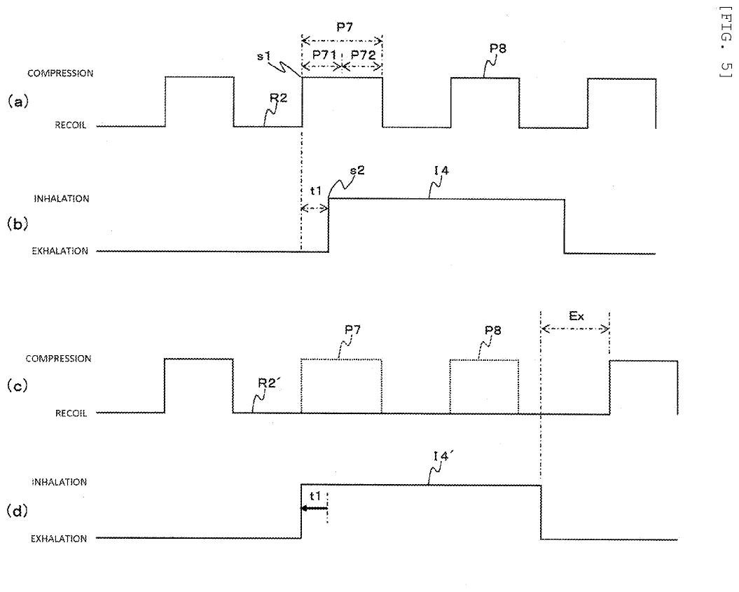

FIGS. 5(a) and 5(b) are waveforms in a case where the sternum compression cycle and the artificial respiration cycle are executed based on setting of the number of times of compression, the number of times of ventilation, and the length of the inhalation period. In FIGS. 5(a) and 5(b), the inhalation period I4 is started during the compression period P7, and the start time s2 of the inhalation period I4 is during the first half period P71 obtained by temporally dividing the compression period P7 into two equal parts. In this state, a part of the compression period P7 and the entire compression period P8 overlap with the inhalation period I4. In this case, as illustrated in FIGS. 5(c) and 5(d), the first control unit preferably hastens start of the inhalation period I4′ by the same time as the time t1 from the start time s1 of the compression period P7 overlapping with the inhalation period I4 to the start time s2 of the inhalation period I4. At this time, the first control unit preferably cancels the entire compression periods P7 and P8 overlapping with the inhalation period I4′ which has been started earlier (indicated by a dotted line in FIG. 5(c)). The first control unit extends the recoil period R2 immediately before the compression period P7 to set the recoil period R2′. As a result, fighting can be avoided even in a case where the inhalation period I4 is started during the compression period P7.

The length of the inhalation period I4′ which has been started earlier is the same as the length of the inhalation period I4 which has been scheduled to be executed before the start time is made earlier. That is, the inhalation period I4′ is temporally shifted forward by the time t1 with respect to the inhalation period I4. In the second example, the number of times of compression is smaller than a set value by the amount overlapping with the inhalation period, and the number of times of ventilation fluctuates with respect to a set value by the amount of shift. The variation in the number of times of ventilation is preferably within ±5% of a set value.

### Third Example

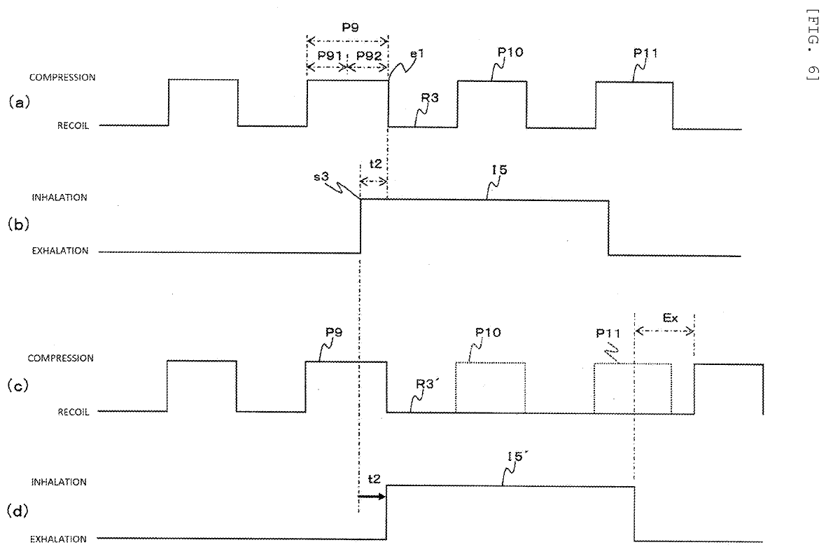

FIGS. 6(a) and 6(b) are waveforms in a case where the sternum compression cycle and the artificial respiration cycle are executed based on setting of the number of times of compression, the number of times of ventilation, and the length of the inhalation period. In FIGS. 6(a) and 6(b), the inhalation period I5 is started during the compression period P9, and the start time s3 of the inhalation period I5 is during the second half period P92 obtained by temporally dividing the compression period P9 into two equal parts. In this state, a part of the compression periods P9 and P11 and the entire compression period P10 overlap with the inhalation period I5. In this case, as illustrated in FIGS. 6(c) and 6(d), the first control unit preferably delays start of the inhalation period I5′ by the same time as the time t2 from the start time s3 of the inhalation period I5 to the end time e1 of the compression period P9 overlapping with the inhalation period I5. At this time, the inhalation period I5′ is started after the end of the compression period P9, and the compression period P9 is executed because the compression period P9 does not overlap with the inhalation period I5′ the start of which has been delayed. The first control unit preferably cancels the entire compression periods P10 and P11 overlapping with the inhalation period I5′ the start of which has been delayed (indicated by a dotted line in FIG. 6(c)). The first control unit extends the recoil period R3 immediately before the compression period P10 to set the recoil period R3′. As a result, fighting can be avoided even in a case where the inhalation period I5 is started during the compression period P9.

The length of the inhalation period I5′ the start of which has been delayed is the same as the length of the inhalation period I5 which has been scheduled to be executed before the start is delayed. That is, the inhalation period I5′ is temporally shifted backward by the time t2 with respect to the inhalation period I5. In the third example, the number of times of compression is smaller than a set value by the amount overlapping with the inhalation period, and the number of times of ventilation fluctuates with respect to a set value by the amount of shift. The variation in the number of times of ventilation is preferably within ±5% of a set value.

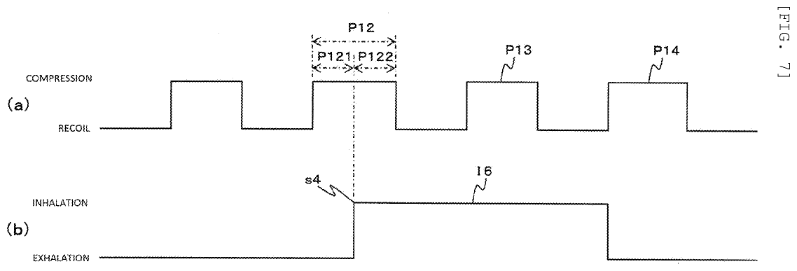

As illustrated in FIGS. 7(a) and 7(b), in a case where the inhalation period I6 is started during the compression period P12 and the start time s4 of the inhalation period I6 is at a boundary between the first half period P121 and the second half period P122 obtained by temporally dividing the compression period P12 into two equal parts, the first control unit may shift the inhalation period I6 forward as in the second example to cancel the entire compression periods P12 and P13 overlapping with the inhalation period (not illustrated) which has been shifted forward, or may shift the inhalation period I6 backward as in the third example to cancel the entire compression periods P13 and P14 overlapping with the inhalation period (not illustrated) which has been shifted backward.

In the first to third examples, as illustrated in FIGS. 4(c) and 4(d), FIGS. 5(c) and 5(d), and FIGS. 6(c) and 6(d), the first control unit preferably restarts the sternum compression cycle a predetermined time after the end of each of the inhalation periods I3, I4′, and I5′, and the sternum compression cycle restarted is preferably started from the compression period. In other words, the first control unit preferably ends the extended recoil periods R1′, R2′, and R3′ after the inhalation periods I3, I4′, and I5′ are ended, respectively. By separating the impact hammer 121 from the chest after the inhalation periods I3, I4′, and I5′ are ended, exhalation time Ex can be secured to prevent an intrathoracic pressure from becoming too high. The length of the exhalation time Ex is not particularly limited, and for example, as compared with a recoil period determined based on setting of the number of times of ventilation and the length of the inhalation period, the length of the exhalation time Ex may be the same as the length of the recoil period, may be longer or shorter than the length of the recoil period, or may be longer than the length of the recoil period.

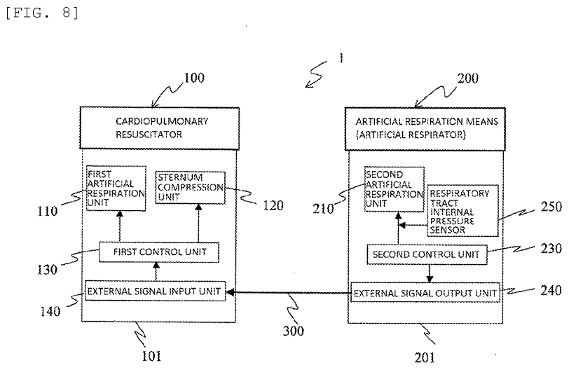

FIG. 8 is a conceptual diagram of another example of the cardiopulmonary resuscitation system according to the present embodiment. Up to this point, the form in which the artificial respiration means (first artificial respiration unit) 110 and the sternum compression means (sternum compression unit) 120 are formed into an integral device as illustrated in FIG. 1 has been described as an example, but the present embodiment is not limited thereto. In a cardiopulmonary resuscitation system 1 according to the present embodiment, as illustrated in FIG. 8, the artificial respiration means 200 and the sternum compression means 120 may be configured as individual devices. By using the artificial respiration means 200 and the sternum compression means 120 as individual device, more accurate cardiopulmonary resuscitation can be performed.

In FIG. 8, the artificial respiration means 200 is an artificial respirator. An artificial respiration means (hereinafter also referred to as an artificial respirator) 200 is, for example, an artificial respirator ANSWER for emergency transport (registered trademark) (manufactured by Kohken Medical Co., Ltd.). As illustrated in FIG. 8, the artificial respirator 200 includes: a second artificial respiration unit 210 for injecting respiratory gas into a patient; a second control unit 230 for controlling the second artificial respiration unit 210 and generating an external signal including a remote control signal to a cardiopulmonary resuscitator; an external signal output unit 240 for outputting the external signal generated by the second control unit 230 to an outside; a respiratory tract internal pressure sensor 250 for detecting a respiratory tract internal pressure of a patient; and a housing 201 for housing these.

The second artificial respiration unit 210 includes an inhalation hose (not illustrated) for injecting respiratory gas into a patient and a gas supply system (not illustrated) for supplying the respiratory gas. One end of the inhalation hose is connected to a hose insertion port (not illustrated) disposed in the housing 201 of the artificial respirator 200. The other end of the inhalation hose is connected to a mask (not illustrated) attached to a patient or a tracheal intubation tube (not illustrated). The gas supply system of the second artificial respiration unit 210 has the same basic configuration as the gas supply system of the first artificial respiration unit 110. A major difference is that a ventilation solenoid valve is disposed in the middle of the piping in the first artificial respiration unit 110, whereas a flow-controllable valve such as a flow regulating valve is disposed in the middle of the piping in the second artificial respiration unit 210.

The second control unit 230 is, for example, a printed circuit board. The second control unit 230 controls the gas supply system of the second artificial respiration unit 210. The second control unit 230 generates an external signal.

The second control unit 230 adjusts the frequency (number of times of ventilation) of the artificial respiration cycle by the second artificial respiration unit 210. Here, the number of times of ventilation is, for example, 2 to 40 times/minute. The second control unit 230 adjusts the ventilation amount of respiratory gas and the length of an inhalation period. The first artificial respiration unit 110 includes a ventilation solenoid valve, whereas the second artificial respiration unit 210 includes a flow-controllable valve such as a flow regulating valve. Therefore, the ventilation amount of respiratory gas by the second artificial respiration unit 210 can be adjusted in a wider range than the range of the ventilation amount of respiratory gas by the first artificial respiration unit 110, and is, for example, 50 to 3000 ml/time. The inhalation period of respiratory gas by the first artificial respiration unit 110 is automatically switched according to the flow rate of respiratory gas, the number of times of ventilation, and the ventilation amount, whereas the inhalation period of respiratory gas by the second artificial respiration unit 210 can be continuously switched alone, for example within a range of 0.3 to 3.0 seconds. Therefore, the second artificial respiration unit 210 can adjust the flow rate more finely than the first artificial respiration unit 110.

The external signal output unit 240 is, for example, a connection terminal of a cable (not illustrated) or a transmission unit of a wireless signal or the like, and outputs an external signal sent from the second control unit 230.

The respiratory tract internal pressure sensor 250 can detect from a negative pressure to a positive pressure, detects a respiratory tract internal pressure of a patient, and outputs a pressure signal to the second control unit 230.

In FIG. 8, the sternum compression means 120 is a sternum compression unit of the cardiopulmonary resuscitator 100. The cardiopulmonary resuscitator 100 has the same basic configuration as the cardiopulmonary resuscitator illustrated in FIG. 1, for example. In a case where the cardiopulmonary resuscitator 100 cooperates with the artificial respirator 200, as illustrated in FIG. 1, the cardiopulmonary resuscitator 100 preferably includes an external signal input unit 140 for inputting an external signal including a remote control signal instructing the sternum compression unit 120 to perform sternum compression. The external signal input unit 140 is, for example, a connection terminal of a cable (not illustrated) or a reception unit of a wireless signal or the like. An external signal input from the external signal input unit 140 is sent to a first control unit 130. The cardiopulmonary resuscitator 100 and the artificial respirator 200 are connected to each other such that an external signal can be transmitted by a signal transmission means 300. The signal transmission means 300 is, for example, a connection cable or wireless communication. The signal transmission means 300 transmits an external signal from the external signal output unit 240 to the external signal input unit 140.

In the cardiopulmonary resuscitation system 1, the artificial respirator 200 performs artificial respiration, and the cardiopulmonary resuscitator 100 performs only sternum compression. That is, the sternum compression unit 120 and the second artificial respiration unit 210 are in an operable state, and the first artificial respiration unit 110 is in a stopped state. At this time, the signal transmission means 300 makes it possible to transmit an external signal from the artificial respirator 200 to the cardiopulmonary resuscitator 100.

In the cardiopulmonary resuscitation system 1, a control means for controlling the artificial respiration means (artificial respirator) 200 and the sternum compression means (sternum compression unit) 120 are preferably mounted on the artificial respirator 200. By control of the sternum compression unit 120 in addition to the second artificial respiration unit 210 by the second control unit 230, the cardiopulmonary resuscitator 100 can be small and lightweight, and sternum compression can be quickly started in an early stage of critical care. The second control unit 230 generates, for example, an external signal including a remote control signal instructing the sternum compression unit 120 to perform sternum compression. The external signal including the remote control signal is output from the external signal output unit 240 and input to the external signal input unit 140 by the signal transmission means 300. The external signal including the remote control signal input by the external signal input unit 140 is sent to the first control unit 130. Upon input of the remote control signal, the first control unit 130 drives the sternum compression unit 120. In this way, the second control unit 230 remotely controls the sternum compression unit 120. In a case where the pressure detected by the respiratory tract internal pressure sensor 250 is a negative pressure, the second control unit 230 outputs a signal for opening a flow regulating valve (not illustrated) to the flow regulating valve (not illustrated) and supplies respiratory gas from the second artificial respiration unit 210.

Avoidance of fighting by the second control unit 230 is performed in a similar manner to those in FIGS. 4 to 6.

Up to this point, the form in which the elevating means 122 of the sternum compression means 120 is a gas driving system has been described, but the present disclosure is not limited to the driving system of the elevating means 122. The driving system of the elevating means 122 may be, for example, an electric driving system or a mixed driving system of a gas driving system and an electric driving system. In a case where the driving system of the elevating means 122 is a gas driving system, according to the classification of the medical apparatus law or the like, the sternum compression means 120 may also be referred to as a “mechanical cardiopulmonary artificial resuscitator”. By adopting the gas driving system, the setting width of the sternum compression depth can be wide, and sternum compression by a method with hands can be reproduced. In a case where the driving system of the elevating means 122 is an electric driving system, according to the classification of the medical apparatus law or the like, the sternum compression means 120 may also be referred to as an “electric cardiopulmonary artificial resuscitator”. By adopting the electric driving system, a device can be simpler. The electric driving system is, for example, an internal battery, an external battery, an external power source such as an AC 100 V power source, or a combination thereof. In a case where the electric driving system is adopted as the driving system of the elevating means 122, the impact hammer 121 reciprocates vertically by motor driving, for example.

### REFERENCE SIGNS LIST

1 Cardiopulmonary resuscitation system
10 Arch portion
11 Top surface portion
12 Left and right side surface portions
13 Fixing portion
14 Adjustment knob
15 Mode switching unit
20 Vertical rod
21 Scale
30 Back plate
100 Cardiopulmonary resuscitation system (cardiopulmonary resuscitator)
101 Housing
110 Artificial respiration means (first artificial respiration unit)
111 Hose
112 Hose insertion port
120 Sternum compression means (sternum compression unit)
121 Impact hammer
121a Impact hammer rod
121b Impact head pad
122 Elevating means
123 Cylinder
130 First control unit
140 External signal input unit
200 Artificial respiration means (artificial respirator)
201 Housing
210 Second artificial respiration unit
230 Second control unit
240 External signal output unit
250 Respiratory tract internal pressure sensor
300 Signal transmission unit
901 Execution period
902 Standby period
I1, I2, I3, I4, I4′, I5, I5′, and I6 Inhalation period
Ex Exhalation time
P1, P2, P3, P4, P5, P6, P7, P8, P9, P10, P11, P12, P12, P13, and P14 Compression period
P71, P91, P121 First half period of compression period
P71, P92, P122 Second half period of compression period
R1, R1′, R2, R2′, R3, and R3′ Recoil period
s1 Start time of compression period
s2, s3, and s4 Start time of inhalation period
e1 End time of compression period
t1, t2 Time

What is claimed is:

1. A cardiopulmonary resuscitation system comprising:
a sternum compression means that includes an impact hammer for compressing the chest of a patient and repeats a sternum compression cycle having, as one cycle, a compression period in which the impact hammer is pressed against the chest and a recoil period in which the impact hammer is separated from the chest;
an artificial respiration means that repeats an artificial respiration cycle having, as one cycle, an inhalation period in which respiratory gas is supplied to the patient and an exhalation period in which supply of the respiratory gas is stopped; and
a control means that controls the artificial respiration means and the sternum compression means, wherein
the control means executes the artificial respiration cycle a predetermined number of times per unit time while executing the sternum compression cycle a predetermined number of times per unit time, and stops pressing the impact hammer against the chest during the compression period overlapping with the inhalation period.

2. The cardiopulmonary resuscitation system according to claim 1, wherein
in a case where the inhalation period is started during the recoil period,
the control means extends the recoil period executed at the start time of the inhalation period at least until the inhalation period ends.

3. The cardiopulmonary resuscitation system according to claim 1, wherein
in a case where the inhalation period is started during the compression period and the start time of the inhalation period is in the first half period obtained by temporally dividing the compression period into two equal parts,
the control means hastens start of the inhalation period by the same time as the time from the start time of the compression period overlapping with the inhalation period to the start time of the inhalation period.

4. The cardiopulmonary resuscitation system according to claim 1, wherein
in a case where the inhalation period is started during the compression period and the start time of the inhalation period is in the second half period obtained by temporally dividing the compression period into two equal parts,
the control means delays start of the inhalation period by the same time as the time from the start time of the inhalation period to the end time of the compression period overlapping with the inhalation period.

5. The cardiopulmonary resuscitation system according to claim 1, wherein
the control means restarts the sternum compression cycle a predetermined time after the end of the inhalation period, and
the sternum compression cycle restarted is started from the compression period.

6. The cardiopulmonary resuscitation system according to claim 1, wherein
the artificial respiration means and the sternum compression means are configured as an integral device.

7. The cardiopulmonary resuscitation system according to claim 1, wherein
the artificial respiration means and the sternum compression means are configured as individual devices.

8. The cardiopulmonary resuscitation system according to claim 7, wherein the control means is mounted on a device constituting the artificial respiration means.

9. The cardiopulmonary resuscitation system according to claim 1, wherein
the sternum compression means includes an elevating means for vertically reciprocating the impact hammer, and
a driving system of the elevating means is a gas driving system, an electric driving system, or a mixed driving system of the gas driving system and the electric driving system.

10. The cardiopulmonary resuscitation system according to claim 2, wherein
the control means restarts the sternum compression cycle a predetermined time after the end of the inhalation period, and
the sternum compression cycle restarted is started from the compression period.

11. The cardiopulmonary resuscitation system according to claim 3, wherein
the control means restarts the sternum compression cycle a predetermined time after the end of the inhalation period, and
the sternum compression cycle restarted is started from the compression period.

12. The cardiopulmonary resuscitation system according to claim 4, wherein
the control means restarts the sternum compression cycle a predetermined time after the end of the inhalation period, and
the sternum compression cycle restarted is started from the compression period.
